(19) United States
(12) Patent Application Publication
(10) Pub. No.: US 2014/0303521 A1
(43) Pub. Date: Oct. 9, 2014

(54) ELECTRONIC DEVICE

(71) Applicant: Sony Corporation, Tokyo (JP)

(72) Inventors: Osamu Nakamura, Saitama (JP); Chika Myoga, Tokyo (JP); Mototsugu Abe, Kanagawa (JP)

(73) Assignee: Sony Corporation, Tokyo (JP)

(21) Appl. No.: 14/355,732

(22) PCT Filed: Oct. 5, 2012
(86) PCT No.: PCT/JP2012/075905

§ 371 Date: May 1, 2014

(30) Foreign Application Priority Data
Nov. 16, 2011 (JP) 2011-250245

(51) Int. Cl. A61B 5/00; A61B 7/00; A61B 7/04

(52) U.S. Cl. CPC A61B 5/6833; A61B 7/04; A61B 7/003
USPC 600/586; 600/300

(57) ABSTRACT

To easily perform replacement of a fixing member for fixing an object on a skin of a human body.

An electronic device includes a biological signal acquisition device, a first sheet, and a second sheet. The biological signal acquisition device acquires a biological signal of the human body in a state of being fixed to the skin of the human body. The first sheet has one surface to be bonded to the biological signal acquisition device and another surface to be bonded to another sheet. Furthermore, a bonding strength per unit area between the other surface of the first sheet and the other sheet is larger than a bonding strength per unit area between the one surface of the first sheet and the biological signal acquisition device. The second sheet includes one surface to be bonded to the other surface of the first sheet and another surface to be bonded to the skin of the human body. Furthermore, the bonding strength per unit area between the one surface of the first sheet and the biological signal acquisition device is larger than a bonding strength per unit area between the other surface of the second sheet and the skin of the human body.

### TECHNICAL FIELD

The present technique relates to an electronic device. Specifically, it relates to an electronic device that acquires a biological signal of a human body in a state of being fixed to the human body.

### BACKGROUND ART

Conventionally, there exists an electronic device that acquires a biological signal such as a respiratory sound, a cardiac sound, a vascular sound, and a vesicular sound of a person by a microphone, which is attached to a skin surface of a human body.

For example, there has been proposed a biological signal collecting device having a sensor unit (sound collection unit), which is stuck to the skin surface of the human body by using an intermediate sheet member provided with a bonding agent layer on both surfaces thereof (see, for example, Patent Document 1).

### CITATION LIST

### Patent Document

Patent Document 1: Japanese Patent Application Laid-Open No. 2008-302052

### SUMMARY OF THE INVENTION

### Problems to be Solved by the Invention

In the above-described conventional technique, with regard to bonding strength of a bonding agent for sticking an intermediate sheet member and the sensor unit, the bonding strength is set to be larger on the sensor unit side than on the intermediate sheet member side. Therefore, in a case where the sensor unit is taken off from the skin surface of the human body after acquisition of the biological signal is completed, a bonding agent layer of the intermediate sheet member remains on the sensor unit when the intermediate sheet member is taken off from the sensor unit.

Here, in general, when the bonding agent is repeatedly stuck and peeled, the bonding strength thereof tends to decrease. Therefore, in order to prevent the sensor unit from unexpectedly peeling off during use, it is preferable that the intermediate sheet member be renewed every time the sensor unit is used. In this case, when the bonding agent layer remains on the sensor unit, as described above, there is a possibility that the intermediate sheet member may not be bonded appropriately with the sensor unit.

The present technique has been devised in view of such situation, and an objective thereof is to enable an easy replacement of a fixing member for fixing an object on the skin of the human body.

### Solutions to Problems

The present technique has been devised for resolving the above-described problem, and a first aspect thereof is an electronic device including: a biological signal acquisition device configured to acquire a biological signal of a human body in a state of being fixed to a skin of the human body; a first sheet having one surface to be bonded to the biological signal acquisition device and another surface to be bonded to another sheet, in which a bonding strength per unit area between the other surface and the other sheet is larger than a bonding strength per unit area between the one surface and the biological signal acquisition device; and a second sheet having one surface to be bonded to the other surface of the first sheet and another surface to be bonded to the skin of the human body, in which the bonding strength per unit area between the one surface of the first sheet and the biological signal acquisition device is larger than a bonding strength per unit area between the other surface and the skin of the human body. Accordingly, it has an effect of fixing the biological signal acquisition device to the skin of the human body by making the bonding strength per unit area between the other surface of the first sheet and the second sheet larger than the bonding strength per unit area between the one surface of the first sheet and the biological signal acquisition device, and by making the bonding strength per unit area between the one surface of the first sheet and the biological signal acquisition device larger than the bonding strength per unit area between the other surface of the second sheet and the skin of the human body.

In the first aspect, an adhesive material is provided on each of the one surface of the first sheet, the other surface of the first sheet, and the other surface of the second sheet. An adhesive strength per unit area of the adhesive material according to the other surface of the first sheet may be larger than an adhesive strength per unit area of an adhesive material according to the one surface of the first sheet, and the adhesive strength per unit area of the adhesive material according to the one surface of the first sheet may be larger than the adhesive strength per unit area of the adhesive material according to the other surface of the second sheet. Accordingly, it has an effect of fixing the biological signal acquisition device to the skin of the human body by making the adhesive strength per unit area of the adhesive material according to the other surface of the first sheet larger than the adhesive strength per unit area of the adhesive material according to the one surface of the first sheet, and by making the adhesive strength per unit area of the adhesive material according to the one surface of the first sheet larger than the adhesive strength per unit area of the adhesive material according to the other surface of the second sheet.

In the first aspect, the above-described first sheet may be formed by bonding the adhesive material according to the above-described one surface of the above-described first sheet and the adhesive material according to the above-described other surface of the above-described first sheet together. Accordingly, it has an effect of fixing the biological signal acquisition device to the skin of the human body by using one sheet formed by bonding the adhesive material according to the one surface and the adhesive material according to the other surface together.

In the first aspect, the above-described first sheet may include a sheet member having one surface, to which the adhesive material according to the above-described one surface of the above-described first sheet is to be bonded, and another surface, to which the adhesive material according to the above-described other surface of the above-described first sheet is to be bonded. Accordingly, it has an effect of fixing the biological signal acquisition device to the skin of the human body by using the first sheet having the sheet member.

In the first aspect, the above-described biological signal acquisition device includes: a bonding surface to be bonded to the above-described one surface of the above-described first sheet; a recessed part provided at the center of the above-described bonding surface; and a biological signal acquisition unit provided at the bottom of the recessed part and configured to acquire the above-described biological signal. The first sheet has a hole provided at the center of each of the above-described one surface and the above-described other surface of the above-described first sheet. The above-described electronic device further includes a sealed space formed by the above-described biological signal acquisition unit, the above-described recessed part, the above-described hole, and the above-described one surface of the above-described second sheet in a state where the above-described biological signal acquisition device is bonded to the above-described first sheet and the above-described first sheet is bonded to the above-described second sheet. Accordingly, it has an effect of forming the sealed space by the biological signal acquisition unit, the recessed part, the hole, and the one surface of the second sheet in a state where the first sheet is bonded to the second sheet.

In the first aspect, a periphery of an opening of the above-described recessed part may be substantially the same as a periphery of the above-described hole, and the above-described biological signal acquisition device and the above-described first sheet may be bonded together such that the periphery of the opening of the above-described recessed part substantially corresponds with the periphery of the above-described hole. Accordingly, it has an effect that the biological signal acquisition device is bonded to the first sheet such that the periphery of the opening of the recessed part substantially corresponds with the periphery of the hole.

In the first aspect, the above-described biological signal acquisition unit may be a microphone. Accordingly, it has an effect that the microphone is used as the biological signal acquisition unit.

In the first aspect, an area of each of the above-described one surface and the above-described other surface of the above-described second sheet may be larger than an area of each of the above-described one surface and the above-described other surface of the above-described first sheet. Accordingly, it has an effect that the first sheet is bonded to the second sheet in a state where the area of each of both surfaces of the second sheet is larger than the area of each of both surfaces of the first sheet.

In the first aspect, the above-described biological signal acquisition device may acquire any one of or a combination of a respiratory sound, a cardiac sound, vascular sound, and a vesicular sound as the above-described biological signal. Accordingly, it has an effect that any one of or the combination of the respiratory sound, the cardiac sound, the vascular sound, and the vesicular sound is acquired as the biological signal.

In the first aspect, the above-described second sheet may be an adhesive film dressing member. Accordingly, it has an effect that the adhesive film dressing member is used as the second sheet.

In the first aspect, a color for making each of the above-described first sheet and the above-described second sheet distinguishable is given at least to one of the above-described first sheet and the above-described second sheet. Accordingly, it has an effect that the biological signal acquisition device is fixed to the skin of the human body by using the first sheet and the second sheet, each having a color that makes it distinguishable.

In the first aspect, the color of the above-described second sheet may be semi-transparent. Accordingly, it has an effect that the biological signal acquisition device is fixed to the skin of the human body by using the second sheet, which is to be semi-transparent.

In this first aspect, a grid pattern design may be given to the above-described one surface of the above-described second sheet. Accordingly, it has an effect that the biological signal acquisition device is fixed to the skin of the human body by using the second sheet, which is given the grid pattern design on the one surface thereof.

### Effects of the Invention

According to the present technique, it is possible to enjoy an excellent effect that replacement of a fixing member for fixing an object on a skin of a human body can be easily performed.

### BRIEF DESCRIPTION OF DRAWINGS

FIG. 1 is a view illustrating a simplified usage example of a biological signal acquisition unit 100 according to a first embodiment of the present technique.

FIG. 2 is a sectional view illustrating an exemplary configuration of the biological signal acquisition unit 100 according to the first embodiment of the present technique.

FIGS. 3a and 3b are a sectional view and a side view illustrating an exemplary configuration of a biological signal acquisition device 200 according to the first embodiment of the present technique.

FIGS. 4a and 4b are a sectional view and a side view illustrating an exemplary configuration of a first adhesive sheet 300 according to the first embodiment of the present technique.

FIGS. 5a and 5b are a sectional view and a side view illustrating an exemplary configuration of a second adhesive sheet 400 according to the first embodiment of the present technique.

FIG. 6 is a sectional view illustrating a state during use of the biological signal acquisition unit 100 according to the first embodiment of the present technique.

FIG. 7 is a sectional view illustrating a state after use of the biological signal acquisition unit 100 is completed according to the first embodiment of the present technique.

FIGS. 8a and 8b are a sectional view and a side view illustrating an exemplary configuration of a second adhesive sheet 500 according to the first embodiment of the present technique.

FIG. 9 is a sectional view of an exemplary configuration of a biological signal acquisition unit 110 according to a second embodiment of the present technique.

FIGS. 10a and 10b are a sectional view and a side view of an exemplary configuration of an adhesive sheet 600 according to the second embodiment of the present technique.

FIGS. 11a and 11b are a sectional view and a side view of an exemplary configuration of the adhesive sheet 600 according to the second embodiment of the present technique.

FIG. 12 is a sectional view illustrating a state during use of the biological signal acquisition unit 110 according to the second embodiment of the present technique.

FIGS. 13a and 13b are a sectional view and a side view illustrating an exemplary configuration of an adhesive sheet 700 according to the second embodiment of the present technique.

FIGS. 14a and 14b are a sectional view and a side view illustrating an exemplary configuration of an adhesive sheet 700 according to the second embodiment of the present technique.

FIGS. 15a to 15c are views each illustrating an exemplary electronic device that uses a biological signal acquired by the biological signal acquisition device 200 according to an embodiment of the present technique.

### MODES FOR CARRYING OUT THE INVENTION

Modes for carrying out the present technique (hereinafter, referred to as embodiments) are described herein. Descriptions will be given in the following order.

1. First embodiment (an example of fixing a biological signal acquisition device on a skin surface of a human body by using a first adhesive sheet and a second adhesive sheet)

2. Second embodiment (an example of fixing the biological signal acquisition device on the skin surface of the human body by using one sheet equivalent to the first adhesive sheet and the second adhesive sheet)

3. Application example

### 1. First Embodiment

### Usage Example of the Biological Signal Acquisition Unit

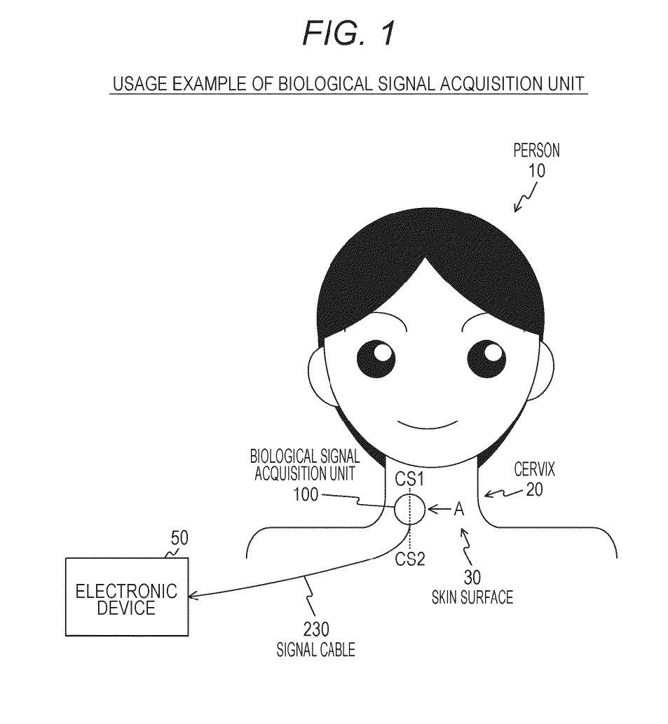

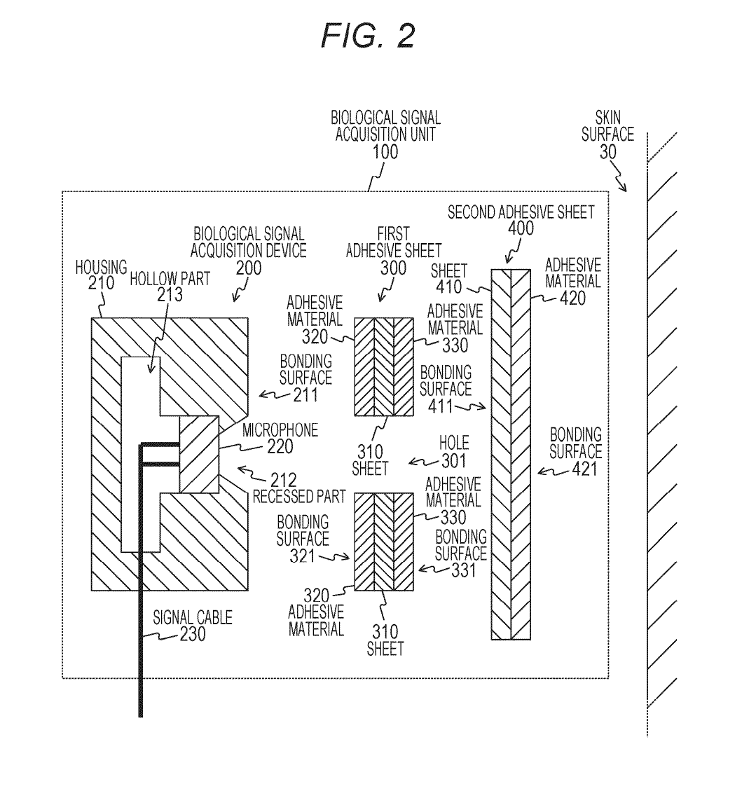

FIG. 1 is a view illustrating a simplified usage example of a biological signal acquisition unit 100 according to the first embodiment of the present technique. Note that a configuration of the biological signal acquisition unit 100 (a cross section of the biological signal acquisition unit 100 viewed from an arrow A (CS1-CS2 cross section)) is described in detail with reference to FIG. 2.

In FIG. 1, a state is illustrated in which the biological signal acquisition unit 100 is attached to a skin surface 30 of a cervix (neck) 20 of a person 10. The person 10 is a target of biological signal acquisition (measurement), and for example, is a patient or a subject.

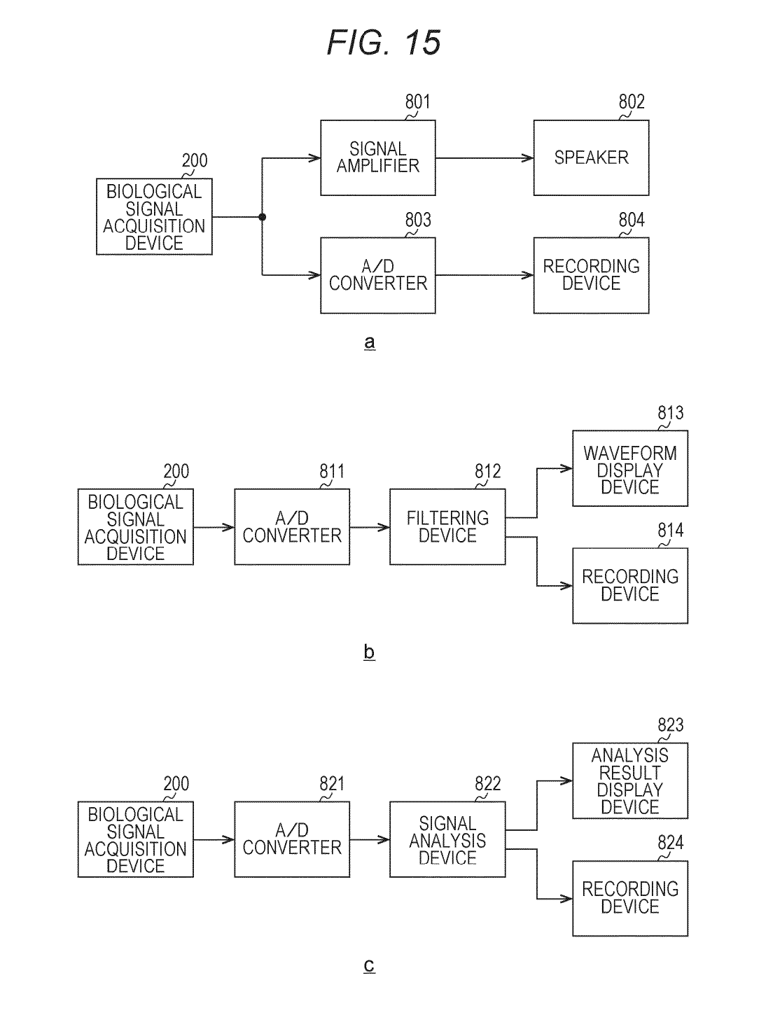

Furthermore, a signal cable 230 provided to the biological signal acquisition unit 100 is connected to an electronic device 50. Note that the electronic device 50 performs various processing (e.g., analog-digital (A/D) conversion processing, signal analysis processing) on a biological signal acquired by the biological signal acquisition unit 100. Note that an exemplary electronic device that performs various processing on the biological signal acquired by the biological signal acquisition unit 100 is described in detail with reference to FIGS. 15a to 15c.

[Exemplary Configuration of the Biological Signal Acquisition Unit]

FIG. 2 is a sectional view illustrating an exemplary configuration of the biological signal acquisition unit 100 according to the first embodiment of the present technique. FIG. 2 is also a sectional view illustrating the cervix 20 of the person 10 with the biological signal acquisition unit 100. That is, in FIG. 2, a relation between the biological signal acquisition unit 100 and the skin surface 30 is illustrated in a simplified manner when the biological signal acquisition unit 100 is not yet attached to the skin surface 30 of the cervix 20 of the person 10.

The biological signal acquisition unit 100 includes a biological signal acquisition device 200, a first adhesive sheet 300, and a second adhesive sheet 400. Note that the biological signal acquisition unit 100 is an example of the electronic device described in the claims.

The biological signal acquisition device 200 includes a housing 210, a microphone 220, and the signal cable 230.

The housing 210 is a cylindrically-shaped housing, and a surface equivalent to one end face of a cylindrical shape is to be a bonding surface 211 to the first adhesive sheet 300. Furthermore, at the center of the bonding surface 211, a conically-shaped recessed part 212 is formed, and the microphone 220 is provided at the bottom of the recessed part 212. Furthermore, a hollow part 213 is formed inside the housing 210, and the signal cable 230 connected to the microphone 220 is output to outside of the housing 210 through the hollow part 213.

Note that in the embodiment of the present technique, a cylindrically-shaped housing is used an example of the housing of the biological signal acquisition device; however, it is also possible to use a housing in a different shape (e.g., polygonal column shape, cone shape).

The microphone 220 is a biological signal acquisition unit that acquires sound as the biological signal. For example, the microphone 220 acquires the sound generated from the skin surface 30 as the biological signal in a state where the biological signal acquisition device 200 is attached to the skin surface 30 of the cervix 20 of the person 10. For example, the microphone 220 acquires any one of or a combination of a respiratory sound, a cardiac sound, a vascular sound, and a vesicular sound as the biological signal. Furthermore, the biological signal acquired by the microphone 220 is output to an external device (e.g., electronic device 50 illustrated in FIG. 1) through the signal cable 230. Note that the microphone 220 is an example of the biological signal acquisition unit described in the claim.

For example, the microphone 220 is realized by an electret capacitor microphone. Note that a microphone other than the electret capacitor microphone (e.g., silicon microphone) may also be used as the biological signal acquisition unit.

Furthermore, although not illustrated or described in detail in the embodiment according to the present technique, a battery for power supply, an A/D converter, and a radio communication device may be incorporated in the biological signal acquisition device 200. Note that the battery for power supply is a battery that supplies power to the biological signal acquisition device 200. Furthermore, the A/D converter is a device that A/D converts the biological signal acquired by the microphone 220. In this way, in a case where the A/D converter is incorporated in the biological signal acquisition device 200, a digital signal converted by the A/D converter can be output from the biological signal acquisition device 200, whereby another A/D converter is not necessary. Furthermore, the radio communication device is a device for wirelessly transmitting the biological signal acquired by the microphone 220 to the external device. For example, it is assumed that a noise may be caused when the signal cable 230 is scraped. Therefore, by incorporating the radio communication device in the biological signal acquisition device 200, it is possible to prevent the noise caused when the signal cable 230 is scraped.

The signal cable 230 is a signal cable for outputting the biological signal acquired by the microphone 220 to the external device.

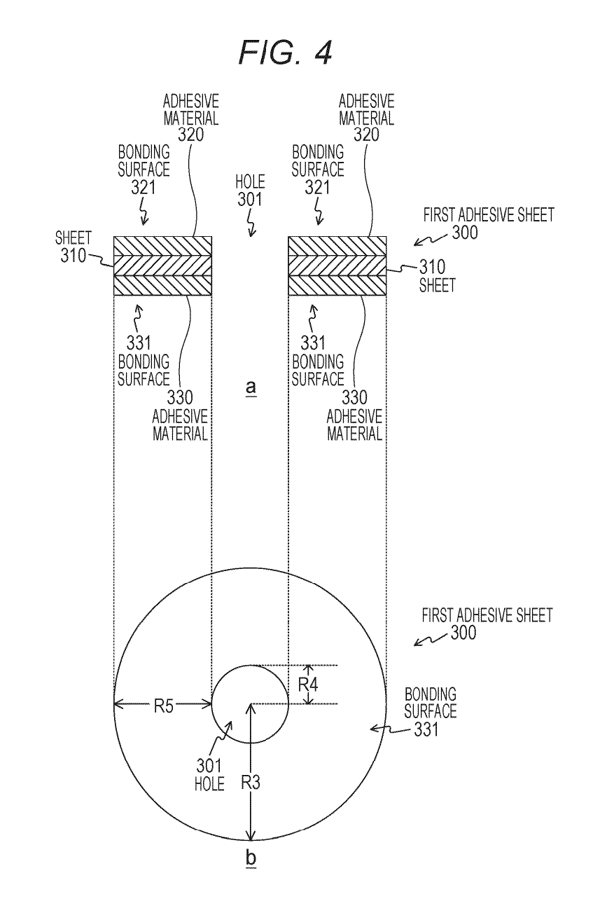

The first adhesive sheet 300 is an annular sheet (as illustrated in FIG. 4b, for example), both of surfaces thereof being bonding surfaces, and includes a sheet 310, an adhesive material 320, and an adhesive material 330. Herein, as illustrated in FIG. 4b, a hole 301 at the center of the first adhesive sheet 300 is circular when viewed from the skin surface 30 side, and a size of a periphery thereof is substantially the same as a size of a periphery of an opening of the recessed part 212 of the biological signal acquisition device 200. Note that the first adhesive sheet 300 is an example of the first sheet described in the claims.

The sheet 310 is an annular sheet member, and the adhesive material 320 is attached to one of surfaces thereof and the adhesive material 330 is attached to the other of the surfaces thereof. As a material of the sheet 310, resin such as polyethylene, a non-woven fabric, and the like may be used.

The adhesive material 320 is an annular adhesive material, one of surfaces thereof being attached to the sheet 310 and the other of the surfaces thereof being a bonding surface 321 to the biological signal acquisition device 200. In a case where the biological signal acquisition device 200 is mounted on the skin surface 30, the bonding surface 321 of the adhesive material 320 is stuck to the bonding surface 211 of the biological signal acquisition device 200.

The adhesive material 330 is an annular adhesive material, one of surfaces thereof being attached to the sheet 310 and the other of the surfaces thereof being a bonding surface 331 to the second adhesive sheet 400. In a case where the biological signal acquisition device 200 is mounted on the skin surface 30, the bonding surface 331 of the adhesive material 330 is stuck to a bonding surface 411 of the second adhesive sheet 400.

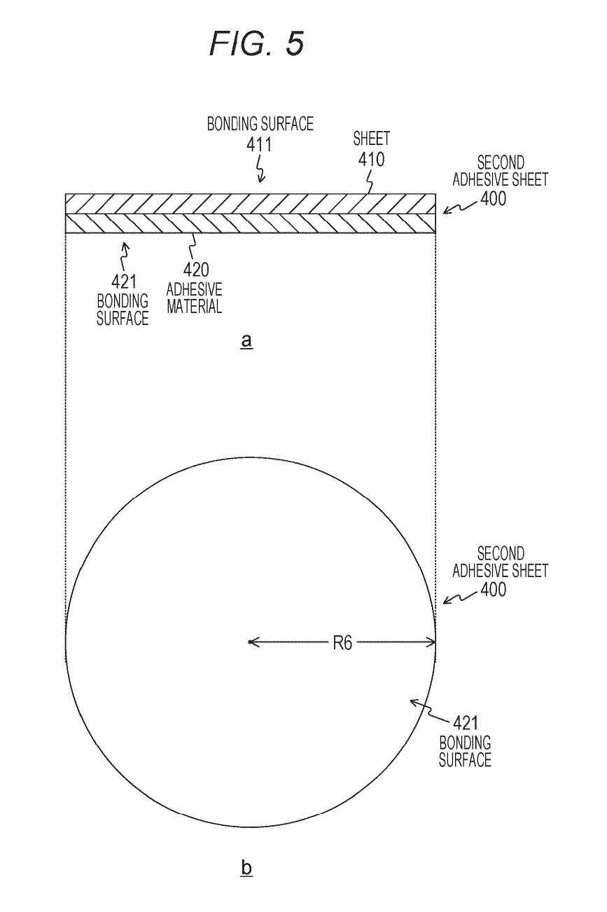

The second adhesive sheet 400 is a circular sheet (as illustrated in FIG. 5b, for example), one of surfaces thereof being an adhesive surface, and includes a sheet 410 and an adhesive material 420.

The sheet 410 is a circular sheet, one of surfaces thereof being attached to the adhesive material 420 and the other of the surfaces thereof being a bonding surface 411 to the first adhesive sheet 300. As a material of the sheet 410, it is preferable that a material capable of efficiently transmitting a vibration of the skin surface 30 to the microphone 220 be used. For example, acrylic resin, polyurethane, nylon, and the like may be used as the material of the sheet 410. Furthermore, by using an elastic sheet such that it can adapt to movement of the skin surface 30, it is possible to decrease damage on the skin of the person 10. As a sheet having this feature, an adhesive film dressing member for medical use, for example, may be used. Note that the dressing material refers to a member used for protecting the skin surface by wrapping or covering it.

The adhesive material 420 is a circular adhesive material, one of surfaces thereof being attached to the sheet 410 and the other of the surfaces being a bonding surface 421 to be bonded to the skin surface 30. In a case where the biological signal acquisition device 200 is mounted on the skin surface 30, the bonding surface 421 of the adhesive material 420 is stuck to the skin surface 30.

In this way, in a case where the biological signal acquisition device 200 is mounted on the skin surface 30, the biological signal acquisition device 200 is stuck to the first adhesive sheet 300 by the adhesive material 320, and the first adhesive sheet 300 is stuck to the second adhesive sheet 400 by the adhesive material 330. Furthermore, the second adhesive sheet 400 is stuck to the skin surface 30 by the adhesive material 420.

[Exemplary Configuration of the Biological Signal Acquisition Device]

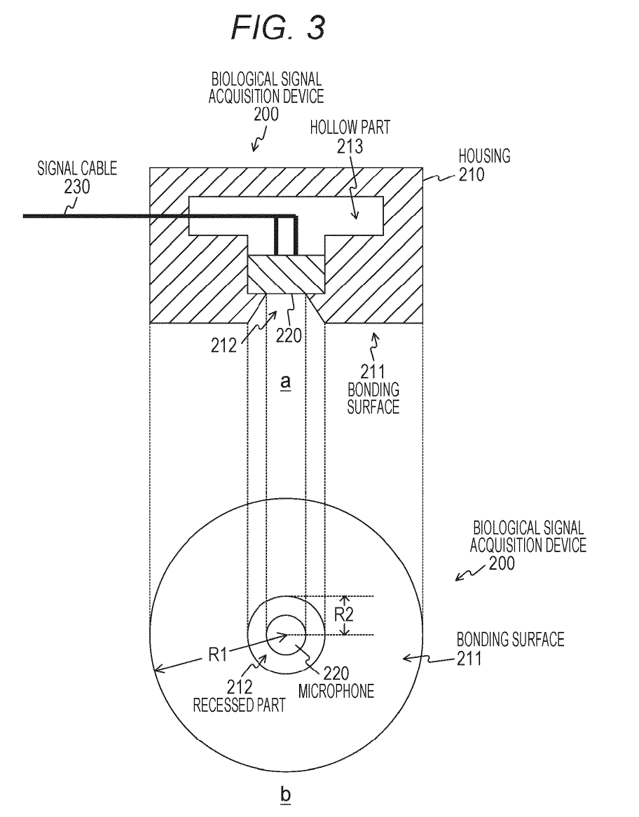

FIGS. 3a and 3b are a sectional view and a side view of an exemplary configuration illustrating the biological signal acquisition device 200 according to the first embodiment of the present technique. That is, the sectional view of the biological signal acquisition device 200 is illustrated in FIG. 3a, and the side view of the biological signal acquisition device 200 (side view viewed from the bonding surface 211 side (skin surface 30 side)) is illustrated in FIG. 3b. Note that the sectional view of the biological signal acquisition device 200 illustrated in FIG. 3a is the same as the sectional view of the biological signal acquisition device 200 illustrated in FIG. 2, except that it is rotated 90 degrees clockwise. Therefore, a detailed description is omitted here.

As described above, the conically-shaped recessed part 212 is formed at the center of the bonding surface 211, and the microphone 220 is provided at the bottom of the recessed part 212.

Here, when R1 is a radius of a circle corresponding to the bonding surface 211 including the recessed part 212, R2 is a radius of a circle corresponding to the opening of the recessed part 212, and A1 is an area of the bonding surface 211, the following formula is established.

A1=π(R1)²−π(R2)²

[Exemplary Configuration of the First Adhesive Sheet]

FIGS. 4a and 4b are a sectional view and a side view illustrating an exemplary configuration of the first adhesive sheet 300 according to the first embodiment of the present technique. That is, FIG. 4a is the sectional view of the first adhesive sheet 300, and FIG. 4b is the side view (side view viewed from the bonding surface 331 side (skin surface 30 side) of the first adhesive sheet 300. Note that the sectional view of the first adhesive sheet 300 illustrated in FIG. 4a is the same as the sectional view of the first adhesive sheet 300 illustrated in FIG. 2, except that it is rotated 90 degrees clockwise. Therefore, a detailed description is omitted here.

As described above, the cylindrically-shaped hole 301 is formed in the first adhesive sheet 300. Here, a size of an outer periphery of the circle corresponding to the bonding surface 211 of the biological signal acquisition device 200 and a size of an outer periphery of a circle corresponding to the first adhesive sheet 300 are the same. Furthermore, a size of an outer periphery of the circle corresponding to the opening of the recessed part 212 and a size of an outer periphery of a circle corresponding to the hole 301 of the first adhesive sheet 300 are the same.

Therefore, when R3 is a radius of the circle corresponding to the first adhesive sheet 300, R4 is a radius of the circle corresponding to the hole 301 of the first adhesive sheet 300, and A2 is an area of the bonding surface 331, R1=R3, R2=R4, and A1=A2. Therefore, the following formula is established.

A2=π(R3)²−π(R4)²

Furthermore, when R5 is a length in a radial direction of the bonding surface 331, the following formula is established.

R5=R3−R4

[Exemplary Configuration of the Second Adhesive Sheet]

FIGS. 5a and 5b are a sectional view and a side view illustrating an exemplary configuration of the second adhesive sheet 400 according to the first embodiment of the present technique. That is, the sectional view of the second adhesive sheet 400 is illustrated in FIG. 5a, and the side view of the second adhesive sheet 400 (the side view when viewed from the bonding surface 421 side (skin surface 30 side)) is illustrated in FIG. 5b. Note that, the sectional view of the second adhesive sheet 400 illustrated in FIG. 5a is the same as the sectional view of the second adhesive sheet 400 illustrated in FIG. 2, except that it is rotated 90 degrees clockwise. Therefore, a detailed description is omitted here.

As described above, unlike the first adhesive sheet 300, there is no hole formed in the second adhesive sheet 400. Furthermore, a size of an outer periphery of a circle corresponding to the bonding surface 421 of the second adhesive sheet 400 is larger than a size of an outer periphery of the circle corresponding to the first adhesive sheet 300.

Therefore, when R6 is a radius of the circle corresponding to the second adhesive sheet 400, and A3 is an area of the bonding surface 421, R1=R3<R6 and A1=A2<A3. Therefore, the following formula is established.

A3=π(R6)²

Here, the first adhesive sheet 300, the second adhesive sheet 400, and an adhesive strength between each of the adhesive materials (adhesive materials 320, 330, and 420) are described herein.

For example, a double-sided tape having different adhesive strengths on both surfaces thereof may be used as the first adhesive sheet 300. In a case where this double-sided tape is used, it is preferable that the adhesive strength of one of the surfaces (surface to be stuck to the biological signal acquisition device 200) be at a level such that the adhesive material does not remain on the bonding surface 211 when it is peeled off from the biological signal acquisition device 200. Note, however, that the following relation is established with regard to this adhesive strength.

Furthermore, as the second adhesive sheet 400, for example, it is possible to use a medical film (for example, one-sided tape) being capable of suppressing the skin damage and being thin and soft enough to allow the biological signal such as the respiratory sound to pass through it.

Let a first adhesive strength AS1 be an adhesive strength per unit area between the biological signal acquisition device 200 and the first adhesive sheet 300 (adhesive strength per unit area of the adhesive material 320), and a second adhesive strength AS2 be an adhesive strength per unit area between the second adhesive sheet 400 and the skin surface 30 (adhesive strength per unit area of the adhesive material 420). Furthermore, let a third adhesive strength AS3 be an adhesive strength per unit area between the first adhesive sheet 300 and the second adhesive sheet 400 (adhesive strength per unit area of the adhesive material 330). In this case, the following relation is established among these three adhesive strengths.

AS2<AS1<AS3

That is, it is set such that a bonding strength per unit area between the bonding surface 331 of the first adhesive sheet 300 and the second adhesive sheet 400 is larger than a bonding strength per unit area between the bonding surface 321 of the first adhesive sheet 300 and the biological signal acquisition device 200. Furthermore, it is set such that the bonding strength per unit area between the bonding surface 321 of the first adhesive sheet 300 and the biological signal acquisition device 200 is larger than a bonding strength per unit area between the bonding surface 421 of the second adhesive sheet 400 and the skin surface 30.

Specifically, the second adhesive strength (adhesive strength per unit area of the adhesive material 420) AS2 is set at a level low enough to be able to suppress the skin damage when the second adhesive sheet 400 is peeled off from the skin surface 30. In this case, an area A3 of the bonding surface 421 is set so as to have an area large enough to sufficiently make up for weakness of the second adhesive strength AS2. For example, in a case where the adhesive strength per unit area of the bonding surface 421 is halved, the area A3 of the bonding surface 421 is set to be doubled.

In this way, by setting the area A3 of the bonding surface 421 to be large, it is possible to suppress the second adhesive strength AS2 to be low. Accordingly, it is possible to fix the biological signal acquisition unit 100 stably on the skin surface 30, and when the biological signal acquisition unit 100 is peeled off from the skin surface 30, it is possible to suppress damage to the skin surface 30.

Here, it is assumed that a hole having the same size as the size of the outer periphery of the circle corresponding to the opening of the recessed part 212 of the biological signal acquisition device 200 be provided in the second adhesive sheet 400. In this case, it is assumed that sticking the hole 301 of the first adhesive sheet 300 and the hole of the second adhesive sheet 400 together by accurately aligning positions thereof could be difficult. Therefore, by allowing the second adhesive sheet 400 to be in a shape having no hole, the area A3 of the bonding surface 421 can be made larger. Accordingly, when a user of the biological signal acquisition unit 100 sticks the first adhesive sheet 300 and the second adhesive sheet 400 together, it is possible to easily stick them together without making an exact alignment.

Note that in the first embodiment of the present technique, an example is described in which a size of the outer periphery of the second adhesive sheet 400 is set to be larger than a size of the outer periphery of the first adhesive sheet 300; however, these sizes can also be substantially the same. For example, whereas the hole 301 is formed in the first adhesive sheet 300, no hole is formed in the second adhesive sheet 400. Therefore, even if the size of the outer periphery of the second adhesive sheet 400 and the size of the outer periphery of the first adhesive sheet 300 are the same, an area of the bonding surface of the second adhesive sheet 400 is larger by an area equivalent to the hole 301 of the first adhesive sheet 300. For example, in a case where the biological signal acquisition unit 100 is stuck to a narrow part (for example, a cervix of an infant), it is effective to decrease the size of the outer periphery of the second adhesive sheet 400.

In this way, in the first embodiment of the present technique, each of the areas of the bonding surfaces 411 and 421 of the second adhesive sheet 400 is set to be larger than each of the areas of the bonding surfaces 321 and 331 of the first adhesive sheet 300.

[Exemplary State of Using the Biological Signal Acquisition Unit]

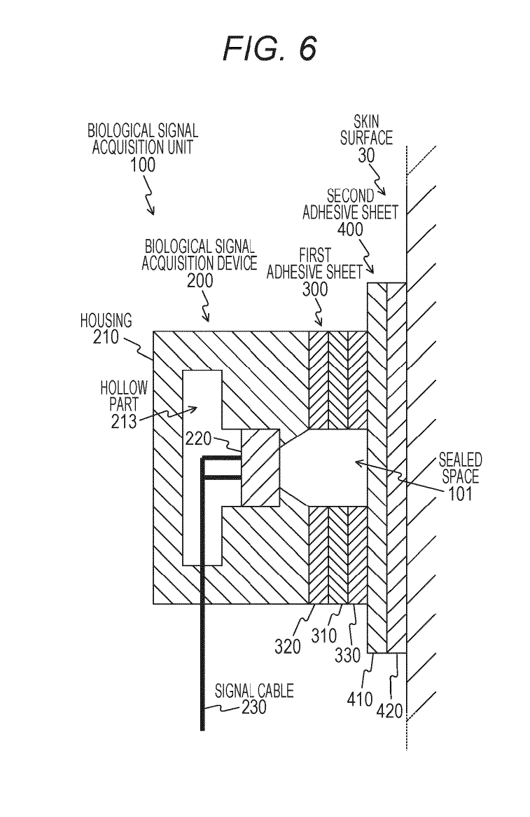

FIG. 6 is a sectional view illustrating a state of using the biological signal acquisition unit 100 according to the first embodiment of the present technique. That is, in FIG. 6, a section is illustrated when the biological signal acquisition unit 100 is mounted on the skin surface 30.

Here, in FIG. 6, the biological signal acquisition unit 100 is illustrated in a state where each of the bonding surfaces 211, 321, 331, and 411 of the biological signal acquisition unit 100 in FIG. 2 is stuck to each other, and the bonding surface 421 is stuck to the skin surface 30.

As illustrated in FIG. 6, the biological signal acquisition device 200 and the first adhesive sheet 300 are bonded together such that a periphery of the opening of the recessed part 212 of the biological signal acquisition device 200 substantially corresponds to a periphery of the hole 301 of the first adhesive sheet 300.

Furthermore, in a state where each of the bonding surfaces 211, 321, 331, and 411 of the biological signal acquisition unit 100 is stuck to each other, a sealed space 101 is formed by the recessed part 212 of the biological signal acquisition device 200 and the hole 301 of the first adhesive sheet 300. That is, the sealed space 101 is a closed space surrounded by the microphone 220 and an internal surface of the recessed part 212 of the biological signal acquisition device 200, an internal surface of the hole 301 of the first adhesive sheet 300, and the bonding surface 411 of the second adhesive sheet 400. In this way, the biological signal acquisition unit 100 is provided with the sealed space 101 formed in a state in which the biological signal acquisition device 200 is bonded to the first adhesive sheet 300, and the first adhesive sheet 300 is bonded to the second adhesive sheet 400.

For example, when a person (for example, subject) 10 respires, a vibration of a gas inside a respiratory tract is transmitted to the skin surface 30. In this way, the vibration transmitted to the skin surface 30 is transmitted to the second adhesive sheet 400, and the vibration transmitted to the second adhesive sheet 400 further causes air inside the sealed space 101 to vibrate. Then, when the vibration of the air inside the sealed space 101 is transmitted to the microphone 220, the vibration of the air is converted into an electric signal by the microphone 220. In this way, the electric signal converted by the microphone 220 is transmitted to the external device (for example, the electronic device 50 illustrated in FIG. 1) through the signal cable 230.

In this way, by causing the air inside the sealed space 101 to vibrate, the vibration of the skin surface 30 is transmitted to the microphone 220; therefore, it is important that the sealed space 101 is hermetically sealed. For example, sound pressure decreases when a gap exists in the sealed space 101, whereby the biological signal acquirable by the microphone 220 may be significantly small.

Therefore, it is necessary to make a length in a radial direction of the first adhesive sheet 300 (R5 illustrated in FIG. 4b) large enough such that there is no partial gap when stuck. For example, it is preferable that the length in the radial direction of the first adhesive sheet 300 (R5 illustrated in FIG. 4b) be 2 mm or more. It is more preferable that it be 3 mm or more.

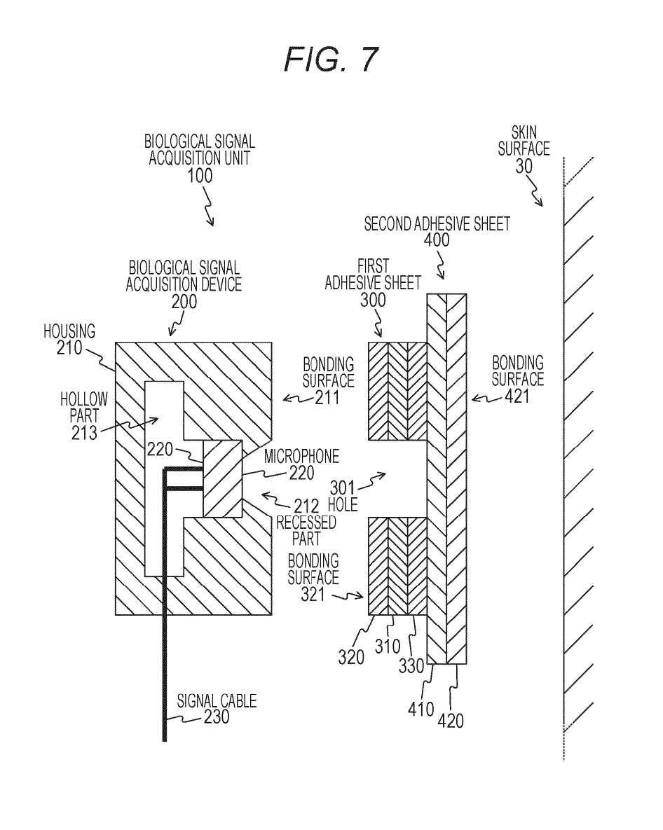

FIG. 7 is a sectional view illustrating a state after use of the biological signal acquisition unit 100 is completed according to the first embodiment of the present technique. That is, in FIG. 7, the section is illustrated in which the biological signal acquisition unit 100 is peeled off from the skin surface 30 after the biological signal of the person 10 has been measured.

For example, in a case where measurement of the biological signal of the person 10 has been completed, the biological signal acquisition unit 100 can be peeled off from the skin surface 30 by pulling the second adhesive sheet 400, which is stuck to the skin surface 30.

Herein, as described above, the third adhesive strength AS3 (adhesive strength per unit area between the first adhesive sheet 300 and the second adhesive sheet 400) is larger than the first adhesive strength AS1 (adhesive strength per unit area between the biological signal acquisition device 200 and the first adhesive sheet 300). Therefore, when the second adhesive sheet 400 is peeled off by pulling the biological signal acquisition device 200, the first adhesive sheet 300 is peeled off from the biological signal acquisition device 200 in a state where it is stuck to the second adhesive sheet 400.

Note that it is also possible to peel off the biological signal acquisition unit 100 from the skin surface 30 in a different way. For example, it is assumed that the first adhesive sheet 300 is peeled off by pulling the biological signal acquisition device 200. In this case, since the third adhesive strength AS3 is larger than the first adhesive strength AS1, the first adhesive sheet 300 is peeled off from the biological signal acquisition device 200 in a state where it is stuck to the second adhesive sheet 400. Then, it can be peeled off from the skin surface 30 by pulling the second adhesive sheet 400 to which the first adhesive sheet 300 is stuck.

In this way, in a case where the biological signal acquisition unit 100 is peeled off from the skin surface 30, since the third adhesive strength AS3 is larger than the first adhesive strength AS1, it is possible to achieve a state in which the first adhesive sheet 300 and the second adhesive sheet 400 are stuck together. Therefore, the first adhesive sheet 300 can be easily peeled off from the biological signal acquisition device 200.

A color of the biological signal acquisition unit 100 is described herein.

For example, by giving a different color to each of the first adhesive sheet 300 and the second adhesive sheet 400, it is possible to easily check existence of each of the adhesive sheets.

For example, it is possible to give a color, which is easily distinguishable from the color of the biological signal acquisition device 200, to the first adhesive sheet 300. In this way, by giving the color to the first adhesive sheet 300, it is possible to let a user of the biological signal acquisition unit 100 easily become aware of forgetting to attach the first adhesive sheet 300 on the biological signal acquisition device 200. Furthermore, when the user of the biological signal acquisition unit 100 forgets to peel off the first adhesive sheet 300 from the biological signal acquisition device 200, it is possible to enable the user to easily notice it. Therefore, a wrong use of the biological signal acquisition unit 100 can be prevented.

Furthermore, to the second adhesive sheet 400, it is possible to give a color easily distinguishable from a color of the skin surface 30, where it is to be stuck. In this way, by giving a color to the second adhesive sheet 400, for example, in a case where the first adhesive sheet 300 is stuck to the second adhesive sheet 400, which is stuck to the skin surface 30, it is possible to easily check a position on the second adhesive sheet 400. Therefore, it is possible to prevent a mistake in sticking the first adhesive sheet 300.

In this way, it is possible to give a color for making each of the first adhesive sheet 300 and the second adhesive sheet 400 distinguishable, at least to one of the first adhesive sheet 300 and the second adhesive sheet 400.

Here, for example, it is assumed that the first adhesive sheet 300 is stuck onto the second adhesive sheet 400, which is stuck to the skin surface 30. In this case, it is assumed that a mistake in sticking may occur by sticking to a position outside of the second adhesive sheet 400, or by sticking to a position where the second adhesive sheet 400 is not stuck (position on the skin surface 30), and the like.

Therefore, for example, it is possible to prevent the mistake in sticking by giving a grid pattern design and the like to the bonding surface 411 of the second adhesive sheet 400. Accordingly, it is possible to easily make an alignment when sticking the first adhesive sheet 300 to the second adhesive sheet 400. Furthermore, by giving a semi-transparent color to the second adhesive sheet 400, it is possible to check a skin condition in a part where the second adhesive sheet 400 is stuck.

[Another Exemplary Configuration of the Second Adhesive Sheet]

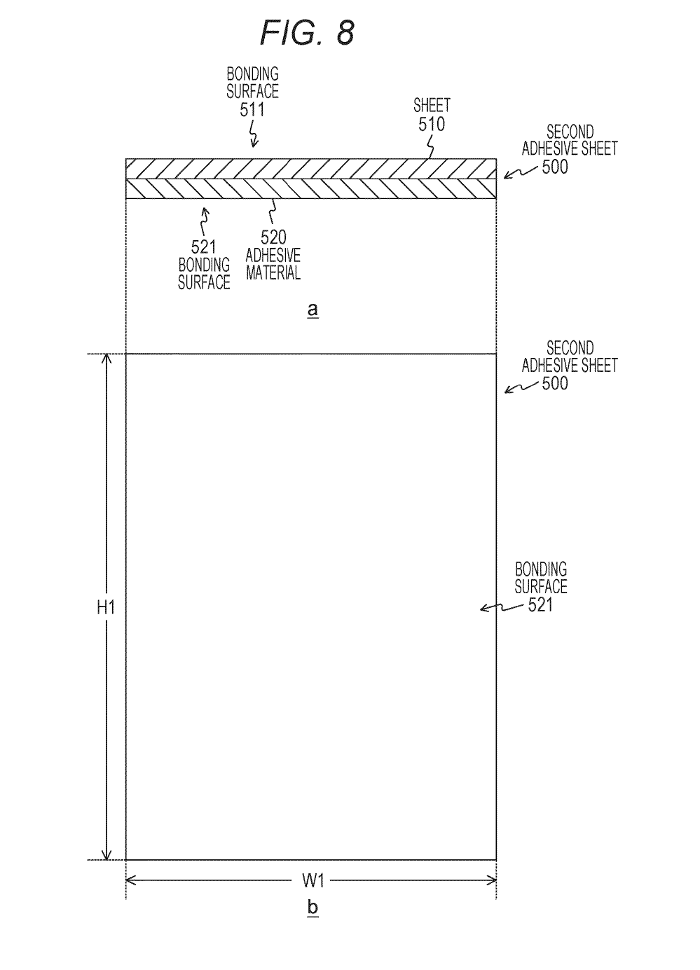

In the descriptions above, the circular second adhesive sheet 400 has been used as an example; however, it is also possible to use a non-circular second adhesive sheet. Therefore, in FIG. 8, an example of the non-circular second adhesive sheet is described.

FIGS. 8a and 8b are a sectional view and a side view illustrating an exemplary configuration of a second adhesive sheet 500 according to the first embodiment of the present technique. That is, the sectional view of the second adhesive sheet 500 is illustrated in FIG. 8a, and the side view of the second adhesive sheet 500 (side view when viewed from a bonding surface 521 side (skin surface 30 side)) is illustrated in FIG. 8b.

Note that the second adhesive sheet 500 is the same as the second adhesive sheet 400 illustrated in FIGS. 5a and 5b, and the like, except that a bonding surface 521 and a bonding surface 511 thereof have a rectangular shape. Therefore, mainly differences from the second adhesive sheet 400 are described herein, and other descriptions are partially omitted.

Here, similarly to the second adhesive sheet 400, no hole is formed in the second adhesive sheet 500. Furthermore, a size of an outer periphery of a rectangle corresponding to the bonding surface 521 of the second adhesive sheet 500 is larger than the size of the outer periphery of the circle corresponding to the first adhesive sheet 300.

Specifically, when H1 is a long side of the rectangle corresponding to the second adhesive sheet 500, W1 is a short side thereof, and A4 is an area of the bonding surface 521, R1=R3≦W1/2≦H1/2 and A1=A2≦A4. Therefore, the following formula is established.

A4=H1×W1

In this way, it is possible to use an adhesive sheet in a different shape as the second adhesive sheet in accordance with a use condition.

### 2. Second Embodiment

In the first embodiment of the present technique, examples are described in which the biological signal acquisition device is fixed to the skin surface of the human body by using the first adhesive sheet and the second adhesive sheet. However, it is also possible to fix the biological signal acquisition device by using one sheet equivalent to the first adhesive sheet and the second adhesive sheet according to the first embodiment of the present technique.

Therefore, in a second embodiment of the present technique, an example is described in which a biological signal acquisition device is fixed by using one sheet equivalent to the first adhesive sheet and the second adhesive sheet according to the first embodiment of the present technique.

[Exemplary Configuration of the Biological Signal Acquisition Unit]

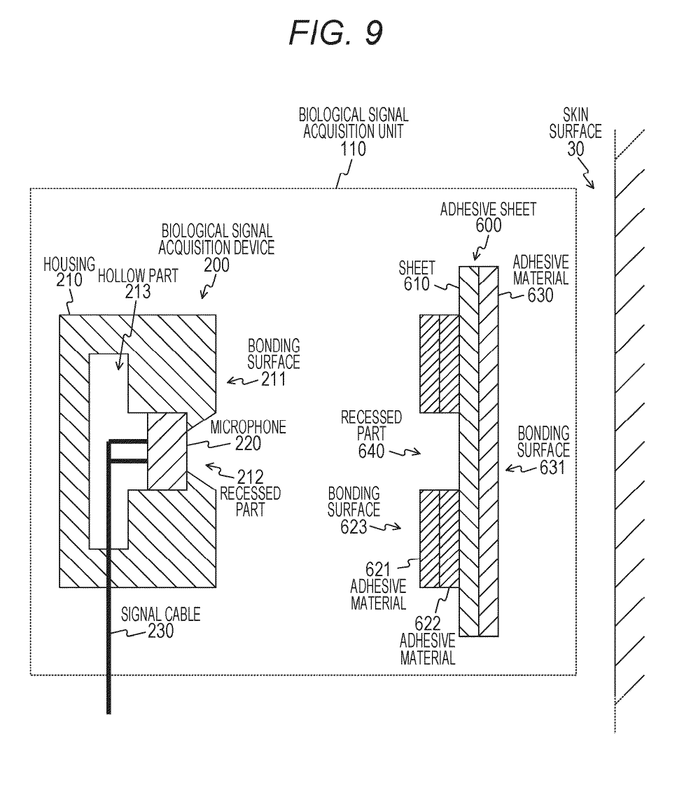

FIG. 9 is a sectional view illustrating an exemplary configuration of a biological signal acquisition unit 110 according to the second embodiment of the present technique. Furthermore, in FIG. 9, a sectional view of a cervix 20 of a person 10 is illustrated with the biological signal acquisition unit 110. That is, in FIG. 9, a relation between the biological signal acquisition unit 110 and a skin surface 30 in a state before the biological signal acquisition unit 110 is attached to the skin surface 30 of the cervix 20 of the person 10 is illustrated in a simplified manner.

The biological signal acquisition unit 110 includes a biological signal acquisition device 200 and an adhesive sheet 600. Note that the biological signal acquisition device 200 is the same as the biological signal acquisition device 200 according to the first embodiment of the present technique. Therefore, a part common with the first embodiment of the present technique is denoted with the same reference numeral, and a description thereof is partially omitted.

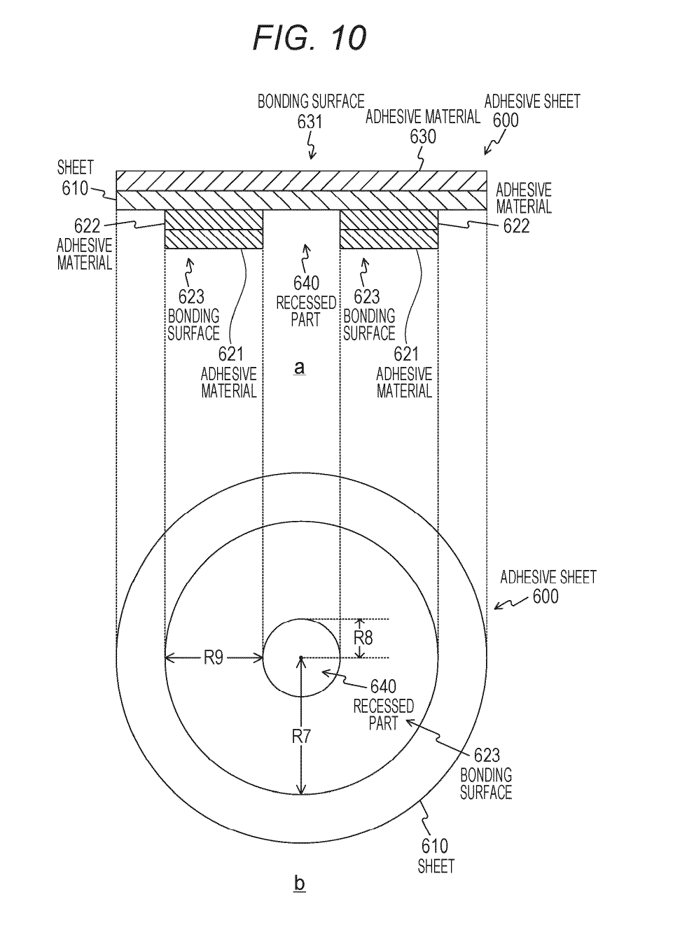

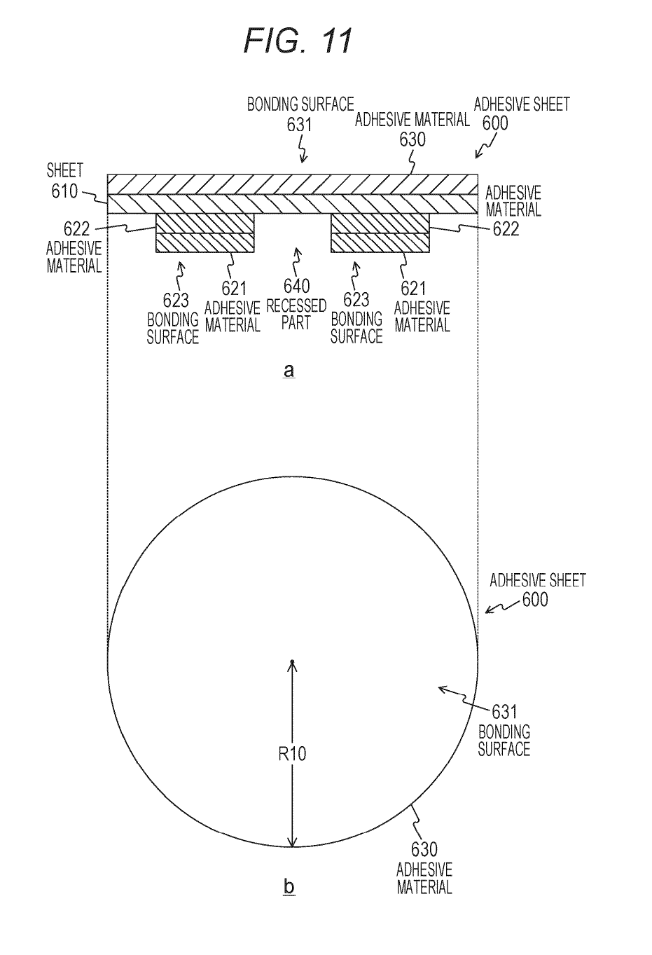

The adhesive sheet 600 is a circular sheet (as illustrated in FIGS. 10b and 11b, for example), both of surfaces thereof being bonding surfaces, and includes a sheet 610, an adhesive material 621, an adhesive material 622, and an adhesive material 630. Here, the sheet 610 is equivalent to the sheet 410 according to the first embodiment of the present technique, and the adhesive material 630 is equivalent to the adhesive material 420 according to the first embodiment of the present technique. Furthermore, the adhesive material 621 is equivalent to the adhesive material 320 according to the first embodiment of the present technique, and the adhesive material 622 is equivalent to the adhesive material 330 according to the first embodiment of the present technique. That is, the sheet 610 and the adhesive material 630 are equivalent to the second adhesive sheet 400 according to the first embodiment of the present technique. Furthermore, the adhesive material 621 and the adhesive material 622 are equivalent to the first adhesive sheet 300 according to the first embodiment of the present technique, except that the sheet 310 is omitted. Therefore, in the second embodiment of the present technique, mainly differences with the first embodiment of the present technique are described, and a description is partially omitted for a common part.

The sheet 610 is a circular sheet, one of surfaces thereof being attached to the adhesive material 630, and the other of the surfaces thereof being attached to the adhesive material 622. Note that, a material and the like of the sheet 610 are similar to those of the sheet 410.

The adhesive material 630 is a circular adhesive material, one of surfaces thereof being attached to the sheet 610 and the other of the surfaces thereof being a bonding surface 631 to be bonded to the skin surface 30. In a case where the biological signal acquisition device 200 is mounted to the skin surface 30, the bonding surface 631 of the adhesive material 630 is stuck to the skin surface 30.

The adhesive material 621 and the adhesive material 622 are annular adhesive materials (illustrated in FIG. 10b, for example) mutually bonded by one surface thereof facing each other, and a recessed part 640 is formed by a hole in the adhesive material 621 and the adhesive material 622. Here, a size of a periphery of an opening of the recessed part 640 is substantially the same as a size of the periphery of the opening of the recessed part 212 of the biological signal acquisition device 200.

Furthermore, the adhesive material 621 has a bonding surface 623, which is a surface on an opposite side of the surface to which the adhesive material 622 is bonded, to the biological signal acquisition device 200. In a case where the biological signal acquisition device 200 is mounted on the skin surface 30, the bonding surface 623 of the adhesive material 621 is stuck to the bonding surface 211 of the biological signal acquisition device 200.

Still further, a surface of the adhesive material 622 on an opposite side of a surface to which the adhesive material 621 is bonded is attached to the sheet 610.

In this way, the sheet formed by mutually bonding the adhesive materials 621 and 622 is equivalent to the first adhesive sheet 300 according to the first embodiment of the present technique.

Furthermore, in a case where the biological signal acquisition device 200 is mounted on the skin surface 30, the biological signal acquisition device 200 and the adhesive sheet 600 are stuck together by the adhesive material 621, and the adhesive sheet 600 and the skin surface 30 are stuck together by the adhesive material 630.

An adhesive strength of each of the adhesive materials (adhesive materials 621, 622, and 630) is described herein. Let a first adhesive strength AS4 be an adhesive strength per unit area between the biological signal acquisition device 200 and the adhesive sheet 600 (adhesive strength per unit area of the adhesive material 621), and let a second adhesive strength AS5 be an adhesive strength per unit area between the adhesive sheet 600 and the skin surface 30 (adhesive strength per unit area of the adhesive material 630). Furthermore, let a third adhesive strength AS6 be an adhesive strength per unit area between the adhesive material 622 and the sheet 610 (adhesive strength per unit area of the adhesive material 622). In this case, the following relation is established among these three adhesive strengths.

AS5<AS4<AS6

[Exemplary Configuration of the Adhesive Sheet]

FIGS. 10 and 11 are a sectional view and a side view illustrating an exemplary configuration of the adhesive sheet 600 according to the second embodiment of the present technique. That is, the sectional views of the adhesive sheet 600 are illustrated in FIGS. 10a and 11a. The side view of the adhesive sheet 600 (side view when viewed from the bonding surface 623 side (biological signal acquisition device 200 side)) is illustrated in FIG. 10b. Furthermore, the side view of the adhesive sheet 600 (side view when viewed from the bonding surface 631 side (skin surface 30 side)) is illustrated in FIG. 11b. Note that sectional views of the adhesive sheet 600 illustrated in FIGS. 10a and 11a are the same as the sectional view of the adhesive sheet 600 illustrated in FIG. 9, except that it is rotated 90 degrees clockwise. Therefore, a detailed description is omitted here.

As illustrated in FIG. 10, the cylindrically-shaped recessed part 640 is formed in the adhesive sheet 600. Here, the size of the outer periphery of the circle corresponding to the bonding surface 211 of the biological signal acquisition device 200 and a size of an outer periphery of a circle corresponding to the adhesive material 621 of the adhesive sheet 600 are the same. Furthermore, the size of the outer periphery of the circle corresponding to the opening of the recessed part 212 and a size of an outer periphery of a circle corresponding to the opening of the recessed part 640 of the adhesive sheet 600 are the same.

Here, when R7 is a radius of the circle corresponding to the adhesive material 621 of the adhesive sheet 600, R8 is a radius of the circle corresponding to the opening of the recessed part 640 of the adhesive sheet 600, and AS is an area of the bonding surface 623, R1=R7, R2=R8, and A1=A5, whereby the following formula is established.

A5=π(R7)²−π(R8)²

Furthermore, when R9 is a length in a radial direction of the bonding surface 623, the following formula is established.

R9=R7−R8

Furthermore, as illustrated in FIGS. 11a and 11b, the recessed part 640 is not formed in a surface on an opposite side of the adhesive sheet 600 (bonding surface 631). Still further, a size of an outer periphery of a circle corresponding to the bonding surface 631 of the adhesive sheet 600 is larger than a size of an outer periphery of a circle corresponding to the adhesive material 621 of the adhesive sheet 600.

Therefore, when R10 is a radius of a circle corresponding to the adhesive material 630 of the adhesive sheet 600, and A6 is an area of the bonding surface 631, R1=R7<R10 and A1=A5<A6. Therefore, the following formula is established.

A6=π(R10)²

Note that a relation between the adhesive strength of each of the adhesive materials (adhesive materials 621, 622, and 630) and the area of each of the bonding surfaces (623 and 631) is substantially the same as the equivalent relationship according to the first embodiment of the present technique; therefore, a description is omitted herein.

[Exemplary State of Using the Biological Signal Acquisition Unit]

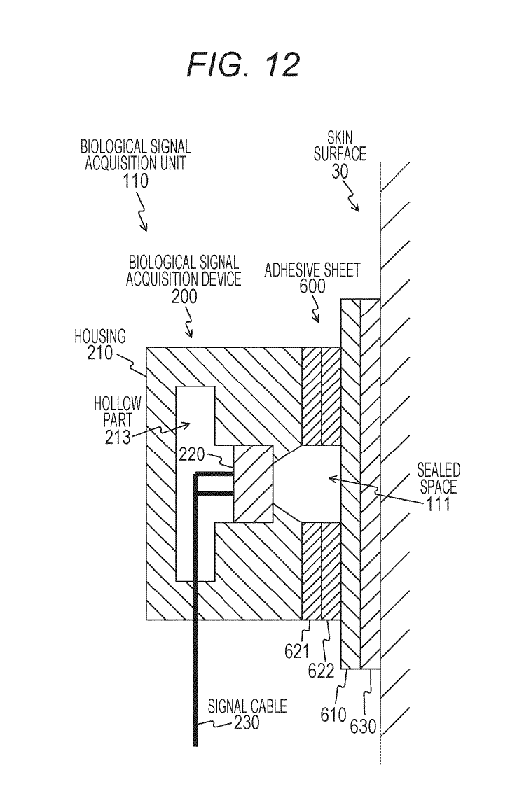

FIG. 12 is a sectional view illustrating a state in which the biological signal acquisition unit 110 according to the second embodiment of the present technique is used. That is, in FIG. 12, the section is illustrated when the biological signal acquisition unit 110 is mounted on the skin surface 30.

Herein, in FIG. 12, the biological signal acquisition unit 110 is illustrated in a state where each of the bonding surfaces 211 and 623 of the biological signal acquisition unit 110 illustrated in FIG. 9 are stuck to each other, and the bonding surface 631 is stuck to the skin surface 30.

Furthermore, in the state where each of the bonding surfaces 211 and 623 of the biological signal acquisition unit 110 is stuck to each other, a sealed space 111 is formed by the recessed part 212 of the biological signal acquisition device 200 and the recessed part 640 of the adhesive sheet 600. That is, the sealed space 111 is a closed space surrounded by the microphone 220 and the internal surface of the recessed part 212 of the biological signal acquisition device 200, and an internal surface and a bottom surface of the recessed part 640 (surface of the sheet 610) of the adhesive sheet 600. Note that the sealed space 111 corresponds to the sealed space 101 according to the first embodiment of the present technique.

Note that acquisition of an electric signal (biological signal) by the microphone 220 by a vibration of air inside the sealed space 111 is the same as the acquisition according to the first embodiment of the present technique; therefore, a description is omitted herein.

Herein, with reference to FIGS. 9 and 12, a case is described in which the biological signal acquisition unit 110 is peeled off from the skin surface 30 after the measurement of the biological signal of the person 10 has been completed.

For example, in a case where the measurement of the biological signal of the person 10 has been completed, the biological signal acquisition unit 110 can be peeled off from the skin surface 30 by pulling the adhesive sheet 600 stuck to the skin surface 30. Then, by pulling the adhesive sheet 600 from the biological signal acquisition device 200, the adhesive sheet 600 can be peeled off from the biological signal acquisition device 200.

Note that it is also possible to peel off the biological signal acquisition unit 110 from the skin surface 30 in a different way. For example, it may be peeled off from the adhesive sheet 600 by pulling the biological signal acquisition device 200. Then, it can be peeled off from the skin surface 30 by pulling the adhesive sheet 600 in a state where the biological signal acquisition device 200 has been peeled off.

Here, as described above, a relation of AS5<AS4<AS6 is established. Therefore, even if operation of pulling the adhesive sheet 600 from the biological signal acquisition device 200 or an operation of directly pulling the biological signal acquisition device 200 stuck to the skin surface 30 is performed, the adhesive materials 621 and 622 and the sheet 610 are not peeled off. Therefore, it is possible to easily peel off the adhesive sheet 600 from the biological signal acquisition device 200.

[Another Exemplary Configuration of Adhesive Sheet]

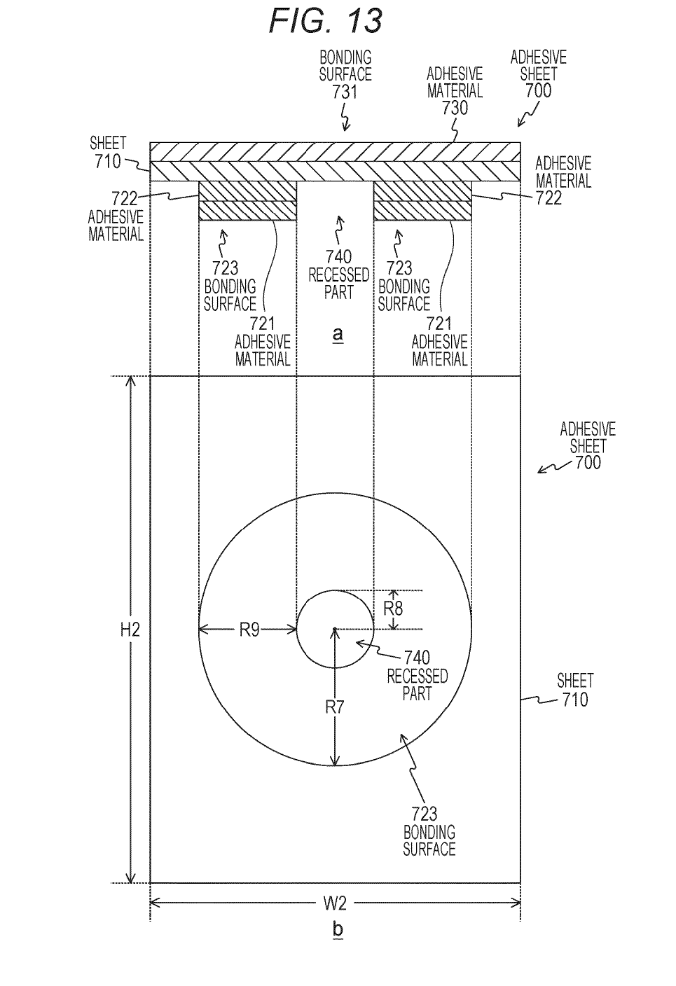

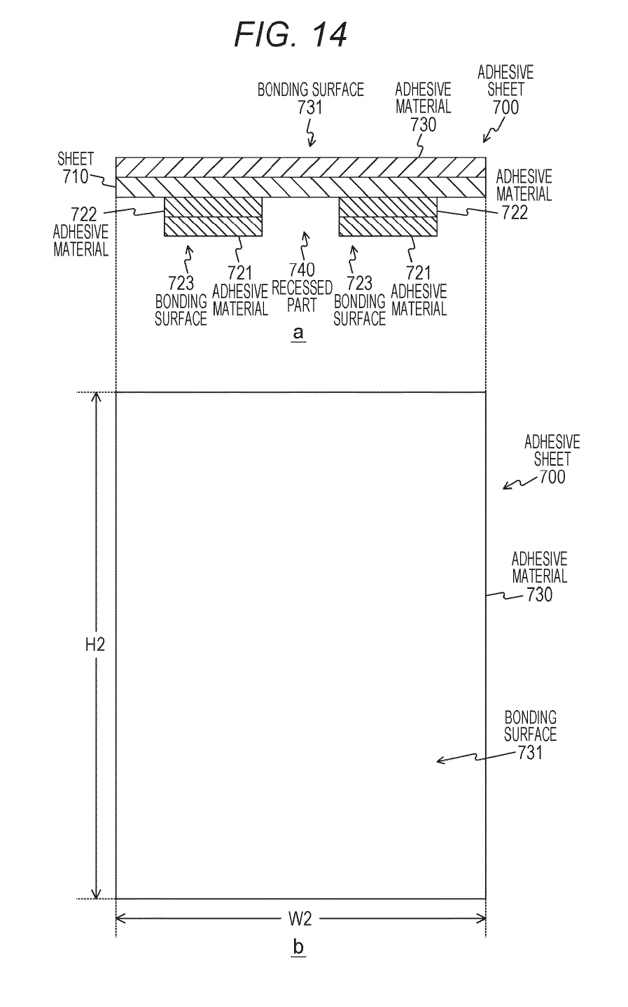

In the descriptions above, the circular adhesive sheet 600 has been used as an example; however, it is also possible to use a non-circular adhesive sheet. Therefore, an example of the non-circular adhesive sheet is described in FIGS. 13 and 14.

FIGS. 13 and 14 are a sectional view and a side view of an exemplary configuration of an adhesive sheet 700 according to the second embodiment of the present technique. That is, the sectional views of the adhesive sheet 700 are illustrated in FIGS. 13a and 14a. The side view of the adhesive sheet 700 (side view when viewed from a bonding surface 723 side (biological signal acquisition device 200 side)) is illustrated in FIG. 13b. Furthermore, the side view of the adhesive sheet 700 (side view when viewed from a bonding surface 731 side (skin surface 30 side)) is illustrated in FIG. 14b.

Note that the adhesive sheet 700 is the same as the adhesive sheet 600 illustrated in FIGS. 10a and 10b, 11a and 11b, and the like, except that the bonding surface 731 (a sheet 710 and an adhesive material 730) has a rectangular shape. Therefore, mainly differences from the adhesive sheet 600 are described herein, and other descriptions are partially omitted.

Here, a size of an outer periphery of a rectangle corresponding to the bonding surface 731 of the adhesive sheet 700 is larger than a size of an outer periphery of a circle corresponding to an adhesive surface 723.

Specifically, when H2 is a long side of the rectangle corresponding to the bonding surface 731 of the adhesive sheet 700, W2 is a short side thereof, and A7 is an area of the bonding surface 731, R1=R7≦W2/2≦H2/2 and A1=A5<A7. Therefore, the following formula is established.

A7=H2×W2

In this way, it is possible to use an adhesive sheet in a different shape as the adhesive sheet in accordance with a use condition.

### 3. Application Example

In the first and the second embodiments of the present technique, an example of fixing the biological signal acquisition unit, which includes the adhesive sheet and the biological signal acquisition device, to the skin surface of the human body has been described. The biological signal acquired in this way can be used after being processed in various ways. Therefore, an example of using the biological signal acquired by the biological signal acquisition unit is described below.

[Example of an Electronic Device Using the Biological Signal]

FIGS. 15a to 15c are views each illustrating an example of an electronic device using the biological signal acquired by the biological signal acquisition device 200 according to the embodiments of the present technique. That is, in FIGS. 15a to 15c, system configurations for monitoring the biological signal (biological signal of a subject) acquired by the biological signal acquisition device 200 are illustrated.

In FIG. 15a, an example of the system configuration in a case where voice output and recording of the biological signal is performed is illustrated.

For example, the biological signal acquired by the biological signal acquisition device 200 is output to a signal amplifier 801 and an A/D converter 803. The signal amplifier 801 amplifies the biological signal output from the biological signal acquisition device 200, outputs it to a speaker 802, and allows the speaker 802 to output it in voice. In this way, by outputting the biological signal acquired by the biological signal acquisition device 200 in voice from the speaker 802, it is possible to hear the biological signal in real time. Note that it is also possible to output a voice from the speaker 802 only at the time of abnormalities.

Furthermore, the A/D converter 803 converts the biological signal (analogue signal) output from the biological signal acquisition device 200 into a digital signal, outputs it to a recording device 804, and allows the recording device 804 to record it. In this way, by recording the biological signal in the recording device 804, it is possible to use the biological signal even after the time of acquisition of the biological signal. Here, an example is described in which the biological signal is output in voice by the speaker 802 and the biological signal is recorded in the recording device 804. Note, however, that it is possible to perform both of the voice output and recording of the biological signal simultaneously in this way or to perform either one of them.

In FIG. 15b, an example of the system configuration in a case where waveform display and recording of the biological signal is performed is illustrated.

For example, the biological signal acquired by the biological signal acquisition device 200 is output to an A/D converter 811. The A/D converter 811 converts the biological signal (analogue signal) output from the biological signal acquisition device 200 into a digital signal, and outputs it to a filtering device 812.

The filtering device 812 performs predetermined filtering processing on the biological signal (digital signal) output from the A/D converter 811. This filtering processing, for example, is processing of attenuating a signal other than the required biological signal. For example, in a case where the required biological signal is a respiratory sound, the signal other than the required biological signal is a cardiac sound and the like. In this way, in a case where the required biological signal is the respiratory sound, processing of attenuating the cardiac sound and the like (signal other than the required biological signal) is performed as the filtering processing.

A waveform display device 813 displays a waveform of the biological signal on which the filtering processing has been performed by the filtering device 812. In this way, by displaying the biological signal on the waveform display device 813, it is possible to easily check a state of the biological signal on a monitor visually.

A recording device 814 records the biological signal on which the filtering processing has been performed by the filtering device 812. In this way, by recording the biological signal on which the filtering processing has been performed in the recording device 814, it is possible to use the biological signal even after the time of acquisition of the biological signal. Here, an example is described in which the biological signal is displayed on the waveform display device 813 and the biological signal is recorded in the recording device 814. It is possible to perform both of wave displaying and recording of the biological signal simultaneously in this way or to perform either one of them.

In FIG. 15c, an example of the system configuration, in a case where signal analysis result display and recording of the biological signal is performed, is illustrated.

For example, the biological signal acquired by the biological signal acquisition device 200 is output to an A/D converter 821. The A/D converter 821 converts the biological signal (analogue signal) output from the biological signal acquisition device 200 into a digital signal, and outputs it to a signal analysis device 822.

The signal analysis device 822 performs predetermined signal analysis processing on the biological signal (digital signal) output from the A/D converter 821. Here, for example, it is assumed that the biological signal to be analyzed is the respiratory sound. In this case, as the signal analysis processing, for example, processing for obtaining the number of respirations per unit time and an interval between respirations is performed. Furthermore, as the signal analysis processing, for example, processing for discriminating between a respiration in a normal state and a respiration in an abnormal state is performed. For example, the respiration in the abnormal state can be determined based on the time passed from a previous respiration. For example, in a case where 20 seconds have passed from the previous respiration, it is determined as being the respiration in the abnormal state (for example, a choking state).

An analysis result display device 823 displays a signal analysis result by the signal analysis device 822. Here, in a case where the analysis result is displayed, it is possible to display an alarm or to notify by an alarm sound in a case where the number of respirations per unit time exceeds a set threshold (for example, an upper limit value or a lower limit value), in a case where the respiration in the abnormal state is detected, or the like. For example, in a case where 20 seconds have passed from the previous respiration, it is determined as being the respiration in the abnormal state, whereby an alarm to notify it is displayed (or, an alarm to notify it is output in voice). Furthermore, in a case where the analysis result is displayed on the analysis result display device 823, since more sophisticated information than the number of respirations and the like can be obtained, a user can concentrate on other important matters. Furthermore, by being notified of the abnormal state by the alarm, it is not necessary for the user to always observe the state of the biological signal, whereby it is possible to improve convenience of the user.

A recording device 824 records the analysis result of the signal analysis processing by the signal analysis device 822. In this way, by recording the analysis result of the signal analysis processing in the recording device 824, the analysis result can be used even after the time of acquisition of the analysis result. Note that an example is described herein in which the analysis result of the signal analysis processing is displayed on the analysis result display device 823, and the analysis result is recorded in the recording device 824. It is possible to perform both of display and recording of the analysis result of the signal analysis processing simultaneously in this way or to perform either one of them.

Note that it may also be a system configuration combining each of the system configurations illustrated in FIGS. 15a to 15c. For example, by combining FIGS. 15a and 15c, it is possible to hear the biological signal in real time as well as to check the abnormal state by the alarm.

Here, conventionally, a stethoscope is widely used for observing a biological sound such as a respiratory sound and a cardiac sound of a patient. In general, the stethoscope includes: a chest piece unit (biological signal acquisition unit) placed on the skin of the patient for picking up an auscultation sound such as the respiratory sound and the cardiac sound; a tube unit for transmitting the auscultation sound picked up by this to an ear piece unit; and an ear piece to be inserted into an ear for hearing the auscultation sound.

Furthermore, in recent years, an electronic stethoscope has been put into practical use, whereby a plurality of persons can hear the auscultation sound simultaneously, or the auscultation sound can be recorded. Furthermore, in an examination and the like of a sleep apnea syndrome, an examination method of monitoring the biological sound (biological signal) continuously for a long time is increasing. Here, in the method of monitoring continuously for a long time, stability of the monitoring is important.

For example, it is not possible to acquire the biological sound (biological signal) when the biological signal acquisition device peels off from the skin. Furthermore, even if it is not peeled off, when the biological signal acquisition device is partially peeled off and is in a state of being loose from a skin surface, there is a possibility that acquisition of the biological sound (biological signal) may become impossible. In this case, even when it is possible to acquire the biological sound (biological signal), it is assumed that it may be a very small sound or that a large surrounding sound (environmental sound) may mix into the biological sound (biological signal).

Therefore, in order to stably monitor the biological sound (biological signal) continuously for a long time, it is necessary that the biological signal acquisition device be securely fixed to the skin. For example, there has been proposed a technique in which a gel sheet is used for fixing the biological signal acquisition device to the skin. In this way, by using the gel sheet, it is possible to secure confidentiality of the sealed space between the microphone incorporated in the biological signal acquisition device and the skin surface as well as to realize difficulty in being peeled off, simultaneously.

In this way, in order to stably monitor the biological sound (biological signal) continuously for a long time, it is important to prevent the biological signal acquisition device from being peeled off from the skin by using a strong adhesive material.

Here, after the biological signal has been acquired by mounting the biological signal acquisition device on the skin of the patient, it is necessary to peel off the biological signal acquisition device from the skin of the patient after the use thereof. However, in a case where the strong adhesive material is used for mounting the biological signal acquisition device, it is important to prevent damage on the skin of the patient when peeling the biological signal acquisition device off. That is, in order to realize stability of monitoring, it is important to prevent the damage on the skin of the patient as well as to prevent the biological signal acquisition device from peeling off from the skin.

Here, in general, when the adhesive material is repeatedly stuck and peeled, the adhesive strength thereof tends to decrease. Therefore, in a case where a frequently-used adhesive material is used for fixing the biological signal acquisition device, there is a possibility that it may be used in a state where the adhesive strength is decreased. In this case, since there is a possibility that the biological signal acquisition device may peel off unexpectedly during use, it is preferable that the adhesive material may be renewed for each use.

However, when the adhesive material is difficult to be peeled from the biological signal acquisition device, it may increase an effort of the user in peeling it off. Furthermore, an unnecessary adhesive material may remain in a part of the biological signal acquisition device. Without noticing it, in a case where a new adhesive material is stuck to the part where the adhesive material remains for acquiring the biological signal, there is a possibility that the biological signal acquisition device may peel off during use. Furthermore, there is a possibility that an accurate biological signal may not be acquired.

As described above, in order to stably monitor the biological signal continuously for a long time, it is necessary to make the biological signal acquisition device difficult to be peeled off from the skin surface. However, when the strong adhesive material is used for fixing the biological signal acquisition device, it is assumed that peeling off of the biological signal acquisition device from the skin may become difficult when the monitoring of the biological signal is completed. That is, in a case where the biological signal acquisition device is taken off from the skin, it is important to prevent the damage on the skin surface. Furthermore, in a case where it is difficult to take off the adhesive material from the biological signal acquisition device, the unnecessarily remaining adhesive material may have an adverse effect on the next monitoring to be performed.

Therefore, in the embodiments of the present technique, an adhesive strength and an area of the adhesive material constituting a fixing member (for example, the first adhesive sheet 300, the second adhesive sheet 400, and the adhesive sheet 600) used for fixing the biological signal acquisition device are set appropriately. Accordingly, it is possible to make it difficult to be peeled from the skin even when used continuously for a long time, and to suppress the damage on the skin when peeling it off from the skin after use. Furthermore, it is possible to easily perform replacement of the fixing member (for example, the first adhesive sheet 300, the second adhesive sheet 400, and the adhesive sheet 600).

Note that in the embodiments of the present technique, the biological signal acquisition device having a cylindrical shape is exemplified as the biological signal acquisition device; however, the embodiments of the present technique are also applicable to the biological signal acquisition device having a shape other than the cylindrical shape (for example, a polygonal column and a cone). Furthermore, in the embodiments of the present technique, the biological signal acquisition device mounted on a cervix for acquiring the biological signal is exemplified; however, the embodiments of the present technique is also applicable to a biological signal acquisition device mounted on another part of a human body (for example, a chest region and an abdominal region) for acquiring the biological signal.

Note that the above-described embodiments are examples for realizing the present technique, and each of matters in the embodiments has a correspondence relation with each of matters used to specify the invention in claims. Similarly, the matters used to specify the invention in claims have the correspondence relation with the matters in the embodiments denoted with the same name. Note, however, that the present technique is not to be limited to the embodiments, and can be realized by adding various modifications on the embodiments without departing from the scope of the present technique.

Note that the present technique may also be configured as below.

(1) An electronic device including:

a biological signal acquisition device configured to acquire a biological signal of a human body in a state of being fixed to a skin of the human body;

a first sheet having one surface to be bonded to the biological signal acquisition device and another surface to be bonded to another sheet, in which a bonding strength per unit area between the other surface and the other sheet is larger than a bonding strength per unit area between the one surface and the biological signal acquisition device; and

a second sheet having one surface to be bonded to the other surface of the first sheet and another surface to be bonded to the skin of the human body, in which the bonding strength per unit area between the one surface of the first sheet and the biological signal acquisition device is larger than a bonding strength per unit area between the other surface and the skin of the human body.

(2) The electronic device according to the above (1), in which

an adhesive material is provided on each of the one surface of the first sheet, the other surface of the first sheet, and the other surface of the second sheet,

an adhesive strength per unit area of an adhesive material according to the other surface of the first sheet is larger than an adhesive strength per unit area of an adhesive material according to the one surface of the first sheet, and

the adhesive strength per unit area of the adhesive material according to the one surface of the first sheet is larger than an adhesive strength per unit area of the adhesive material according to the other surface of the second sheet.

(3) The electronic device according to the above (2) in which the first sheet is formed by bonding the adhesive material according to the one surface of the first sheet and the adhesive material according to the other surface of the first sheet together.

(4) The electronic device according to the above (2), in which the first sheet includes a sheet member having one surface, to which the adhesive material according to the one surface of the first sheet is to be bonded, and another surface, to which the adhesive material according to the other surface of the first sheet is to be bonded.

(5) The electronic device according to any of the above (1) to (4), the biological signal acquisition device including:

a bonding surface to be bonded to the one surface of the first sheet;

a recessed part provided at the center of the bonding surface; and

a biological signal acquisition unit provided at the bottom of the recessed part and configured to acquire the biological signal,

the first sheet having a hole provided at the center of each of the one surface and the other surface of the first sheet,

the electronic device further including a sealed space formed by the biological signal acquisition unit, the recessed part, the hole, and the one surface of the second sheet in a state where the biological signal acquisition device is bonded to the first sheet, and the first sheet is bonded to the second sheet.

(6) The electronic device according to the above (5), in which a periphery of an opening of the recessed part is substantially the same as a periphery of the hole, and

the biological signal acquisition device and the first sheet are bonded together such that the periphery of the opening of the recessed part substantially corresponds with the periphery of the hole.

(7) The electronic device according to the above (5) or (6), in which the biological signal acquisition unit is a microphone.

(8) The electronic device according to any of the above (1) to (7), in which an area of each of the one surface and the other surface of the second sheet is larger than an area of each of the one surface and the other surface of the first sheet.

(9) The electronic device according to any of the above (1) to (8), in which the biological signal acquisition device acquires any one of or a combination of a respiratory sound, a cardiac sound, a vascular sound, and a vesicular sound as the biological signal.

(10) The electronic device according to any of the above (1) to (9), in which the second sheet is an adhesive film dressing member.

(11) The electronic device according to any of the above (1) to (10), in which a color for making each of the first sheet and the second sheet distinguishable is given at least to one of the first sheet and the second sheet.

(12) The electronic device according to any of the above (1) to (10), in which the color of the second sheet is semi-transparent.

(13) The electronic device according to any of (1) to (10), wherein a grid pattern design is given to the one surface of the second sheet.

### REFERENCE SIGNS LIST

10 Person
20 Cervix
30 Skin surface
50 Electronic device
100,110 Biological signal acquisition unit
101,111 Sealed space
200 Biological signal acquisition device
210 Housing
212 Recessed part
213 Hollow part
220 Microphone
230 Signal cable
300 First adhesive sheet
301 Hole
310 Sheet
320,330 Adhesive material
400 Second adhesive sheet
410 Sheet
420 Adhesive material
500 Second adhesive sheet
510 Sheet
520 Adhesive material
600 Adhesive sheet
610 Sheet
621,622,630 Adhesive material
640 Recessed part
700 Adhesive sheet
710 Sheet
721,722,730 Adhesive material
740 Recessed part
801 Signal amplifier
802 Speaker
803,811,821 A/D converter
804,814,824 Recording device
812 Filtering device
813 Waveform display device
822 Signal analysis device
823 Analysis result display device

1. An electronic device comprising:
a biological signal acquisition device configured to acquire a biological signal of a human body in a state of being fixed to a skin of the human body;
a first sheet having one surface to be bonded to the biological signal acquisition device and another surface to be bonded to another sheet, wherein a bonding strength per unit area between the other surface and the other sheet is larger than a bonding strength per unit area between the one surface and the biological signal acquisition device; and
a second sheet having one surface to be bonded to the other surface of the first sheet and another surface to be bonded to the skin of the human body, wherein the bonding strength per unit area between the one surface of the first sheet and the biological signal acquisition device is larger than a bonding strength per unit area between the other surface and the skin of the human body.

2. The electronic device according to claim 1, wherein
an adhesive material is provided on each of the one surface of the first sheet, the other surface of the first sheet, and the other surface of the second sheet,
an adhesive strength per unit area of the adhesive material according to the other surface of the first sheet is larger than an adhesive strength per unit area of an adhesive material according to the one surface of the first sheet, and
the adhesive strength per unit area of the adhesive material according to the one surface of the first sheet is larger than an adhesive strength per unit area of the adhesive material according to the other surface of the second sheet.

3. The electronic device according to claim 2 wherein the first sheet is formed by bonding the adhesive material according to the one surface of the first sheet and the adhesive material according to the other surface of the first sheet together.

4. The electronic device according to claim 2, wherein the first sheet includes a sheet member having one surface, to which the adhesive material according to the one surface of the first sheet is to be bonded, and another surface, to which the adhesive material according to the other surface of the first sheet is to be bonded.

5. The electronic device according to claim 1, the biological signal acquisition device including:
a bonding surface to be bonded to the one surface of the first sheet;
a recessed part provided at the center of the bonding surface; and
a biological signal acquisition unit provided at the bottom of the recessed part and configured to acquire the biological signal,
the first sheet having a hole provided at the center of each of the one surface and the other surface of the first sheet,
the electronic device further comprising a sealed space formed by the biological signal acquisition unit, the recessed part, the hole, and the one surface of the second sheet in a state where the biological signal acquisition device is bonded to the first sheet and the first sheet is bonded to the second sheet.

6. The electronic device according to claim 5, wherein
a periphery of an opening of the recessed part is substantially the same as a periphery of the hole, and
the biological signal acquisition device and the first sheet are bonded together such that the periphery of the opening of the recessed part substantially corresponds with the periphery of the hole.

7. The electronic device according to claim 5, wherein the biological signal acquisition unit is a microphone.

8. The electronic device according to claim 1, wherein an area of each of the one surface and the other surface of the second sheet is larger than an area of each of the one surface and the other surface of the first sheet.

9. The electronic device according to claim 1, wherein the biological signal acquisition device acquires any one of or a combination of a respiratory sound, a cardiac sound, a vascular sound, and a vesicular sound as the biological signal.

10. The electronic device according to claim 1, wherein the second sheet is an adhesive film dressing member.

11. The electronic device according to claim 1, wherein a color for making each of the first sheet and the second sheet distinguishable is given at least to one of the first sheet and the second sheet.

12. The electronic device according to claim 1, wherein a color of the second sheet is semi-transparent.

13. The electronic device according to claim 1, wherein a grid pattern design is given to the one surface of the second sheet.
